(19) United States
(12) Patent Application Publication
(10) Pub. No.: US 2021/0077326 A1
(43) Pub. Date: Mar. 18, 2021

(54) AUTOMATED TURNING SYSTEM FOR AN IMMOBILE PERSON

(71) Applicant: BeacoHealth Pty Ltd, Harrington Park (AU)

(72) Inventors: Yi Xiong GOH, Kuala Lumpur (MY); Tristan Claude BERRY, Harrington Park (AU); Jannatul NAEEM, Kuala Lumpur (MY)

(21) Appl. No.: 16/992,176

(22) Filed: Aug. 13, 2020

(30) Foreign Application Priority Data
Sep. 12, 2019 (MY) PI 2019005256

(51) Int. Cl. A61G 7/00; A61G 7/10

(52) U.S. Cl. CPC A61G 7/001; A61G 7/1019; A61B 5/447; A61G 2203/12; A61G 2203/34; A61G 7/1026

(57) ABSTRACT

The present invention particularly is an automated turning system for an immobile person and is aimed to reduce or avoid bedsores. The invention allows for appropriate turning of the immobile person on a regular schedule as well as when the system detects circumstances where pressure sores are likely to occur. The system can be adapted for use with an existing bed or can be manufactured together with a bed.

The invention relates to a system for prevention and relief of pressure sores. More particularly, the system is an automated system that permits convenient raising and lowering of a person for the prevention and relief of pressure sores.

### DESCRIPTION OF THE PRIOR ART

Persons who must spend extended periods of time in bed are prone to develop decubitus ulcers. Often called “pressure ulcers” or “bedsores”, decubitus ulcers occur over the bony prominences and weight-bearing surfaces of immobile persons.

It is possible to prevent and treat bedsores by providing appropriate turning of an immobile person on a regular schedule. Immobile persons who are not turned regularly (as is the case in many health-care facilities because of inadequate staffing) are at high risk for developing bedsores, which are typically slow to heal and quick to become infected.

Although general medical practice recommends that a person be rotated from one position in bed to another position in bed about every two hours to prevent and treat bedsores, many care facilities and private homes do not reach this standard (turning their patients less often).

At present, there are a number of systems, devices and methods for minimizing pressure sores. An example is the Pressure-Relieving Support Surfaces (PRSS). PRSS minimises the intensity and duration of pressure exposure over vulnerable skin sites, either by redistributing the immobile person's weight over a larger contact area via foam/air/fluid overlays (low-tech constant low pressure), or by mechanically varying the pressure beneath the patient (high-tech alternating pressure mattress).

The use of PRSS in combination with the two hourly body turning has been shown to reduce pressure ulcer incidence, and has been widely adopted in the prevention and treatment of pressure ulcers. However, when used in isolation, PRSS does not significantly reduce the incidence of pressure ulcers.

Standard-style hospital beds that turn a person from side to side in bed typically require human assistance at some point during the turning process as these beds cannot be operated automatically. Further, these beds are bulky, immobile and expensive as the whole bed would have to be purchased by the patient or care facilities.

U.S. Pat. No. 8,528,135 B2 describes a pressure sore relief system which includes a plurality of sensors in communication with a surface, whereby each sensor operates independently of one another for detecting a pressure level at a particular region of the surface and generating a feedback signal that corresponds to the detected pressure level, and a system controller adapted to receive the feedback signals from the sensors and generate a plurality of output signals in response to each of the feedback signals. The pressure sore relief system includes a plurality of actuators in communication with the surface and being adapted to receive the output signals from the system controller, whereby each output signal generated by the system controller is associated with one of the actuators. The actuators are adapted to selectively vibrate for minimizing the likelihood of a patient developing pressure sores.

KR10-1248685 B1 describes an apparatus for the prevention of bedsores comprising a frame and multiple linear actuators. The multiple linear actuators are connected for the network controller which controls the movement of the actuators, and thus moving the frame up and down.

US 2017/0128297 A1 describes a system and method to reduce pressure on selected areas on a body which includes an array of programmable supports and at least one sensor to detect a physical property of the body. Each programmable support of the array of programmable supports includes an adjustable member and a mechanism to adjust the length of the adjustable member based on the detected physical property of the body.

There are also other solutions which use pressure sensors embedded onto overlays to monitor the body surface pressure of immobile persons. These devices subsequently send reminders to healthcare workers when the body surface pressure has exceeded a particular time threshold. The disadvantage of such systems lies in the pressure data which often appears in the form of a visual heat-map, which cannot reliably identify the body surface pressure of specific areas i.e. areas which are at high risk of developing pressure ulcers. Furthermore, this heat-map of pressure values may not accurately depict body positions i.e. the heat-map is not able to show whether the immobile person is lying supine or sideways.

In spite of these advances, there remains a need for a system that is able to not only appropriately turn an immobile person on a regular schedule but to also detect circumstances where pressure sores are likely to occur and automatically respond for minimizing the occurrence of pressure sores.

### SUMMARY OF THE INVENTION

In accordance with an aspect of the invention, there is provided an automated turning system for an immobile person comprising:

a plurality of pressure sensors for measuring pressure forces applied to the immobile person and generating response signals that correspond to said pressure forces applied to said immobile person;
a system controller adapted to receive said response signals from said pressure sensors and generate a plurality of output signals in response to each of said response signals;
a plurality of actuators being adapted to receive said output signals from said system controller; and
a lifting attachment comprising a sheet and a pair of connecting beams, each beam being connected to ones of said plurality of actuators for raising a portion of said sheet to a tilted position, wherein, in use, the immobile person is positioned atop said sheet;
wherein said system controller receives said response signals for detecting whether a pressure threshold value for said immobile person has been exceeded for any of said sensors, and in turn, said system controller transmits said output signals to said actuators for engagement.

The invention thus helps to detect circumstances where pressure sores are likely to occur and automatically responds for minimizing the occurrence of pressure sores.

The invention can be manufactured without an existing bed. This reduces manufacturing costs and allows the apparatus to be sold at a lower cost compared to a bed with the apparatus already mounted on the bed.

In one embodiment, the system further comprises a bed with bed frames, wherein the plurality of actuators is mounted on the bed frame.

The invention can be used on an existing bed without the need to substantially modify the bed. This allows for cost savings.

In a further embodiment, the plurality of actuators is engaged if a predetermined threshold value of the pressure sensor has not been exceeded for any of said plurality of sensors for a preset interval.

The preferred preset interval is two hours.

This prevents the occurrence of pressure sores as general medical practice recommends that a person be rotated from one position in bed to another position in bed about every two hours.

In another embodiment, each of said pressure sensor operates independently of one another.

In another embodiment, the plurality of pressure sensors operates cohesively as a singular unit.

In an embodiment, the pressure forces are those that are detected in any one or more of the occipital region, the thoracic spine region, the shoulder blades region, the sacral region and the calcaneal region.

In another embodiment, the pressure sensors are embedded into the turning sheet.

In a further embodiment, the pressure sensors are embedded into a wearable article.

The use of wearables helps to address many issues as wearables are a relatively low cost technology that can collect masses of data continuously in a substantially efficient and easy to use process.

In one embodiment, the wearable article is any one or more of a headband, vest, belt or socks.

More than one wearable article can be worn at one time. This allows monitoring and detection of pressure sores in more than one region, and consequently, minimizing the occurrence of pressure sores.

In a further embodiment, the pressure sensors are embedded into a disposable patch.

This is advantageous for short-term bedridden patients in healthcare facilities as it would be cheaper to dispose of the patches after each patient use compared to having to wash, dry and sterilize the wearable articles after each patient use.

In another embodiment, the plurality of pressure sensors are wirelessly connected to the system controller.

In a further embodiment, the plurality of actuators are connected to the system controller via a wired connection.

In an embodiment, said plurality of pressure sensors are Force Sensitive Resistors.

In an embodiment, a bed comprises of the automated turning system.

Additional advantages of the system of this invention will be elaborated in the following pages.

### BRIEF DESCRIPTION OF THE DRAWINGS

FIG. 1A shows a perspective view of a bed including a mattress having a pressure sore relief system and a system controller, in accordance with one embodiment of the present invention.

FIG. 1B shows a perspective view of a bed including a turning sheet having a pressure sore relief system, in accordance with one embodiment of the present invention.

FIG. 1C shows the connection between the ends of a connecting beam and an actuator.

FIG. 1D shows the connection between the actuator and the bed frame.

FIG. 1E shows a perspective view of a bed when actuators are engaged, in accordance with one embodiment of the present invention.

FIG. 2A shows a wearable article in the form of a belt.

FIG. 2B shows a system to reduce pressure on the sacral region.

FIG. 3 shows a wearable article in the form of a headband worn by an immobile person.

FIG. 4 shows a wearable article in the form of a vest.

FIG. 5 shows a wearable article in the form of a pair of socks.

FIG. 6 shows a pressure sensor in the form of a sensor patch.

FIG. 7 shows a block diagram of the system depicted in FIG. 1.

### DETAILED DESCRIPTION OF THE EMBODIMENTS

The present invention particularly is an automated patient turning system which is aimed to reduce or avoid bedsores in patients that are bedridden. The invention allows for appropriate turning of an immobile person on a regular schedule as well as when the system detects circumstances where pressure sores are likely to occur. The system can be used with an existing bed or can be manufactured together with a bed.

An immobile person is a person who is limited in their ability to change positions, for example, those who have recently had surgery or those who spend most of their time in a bed.

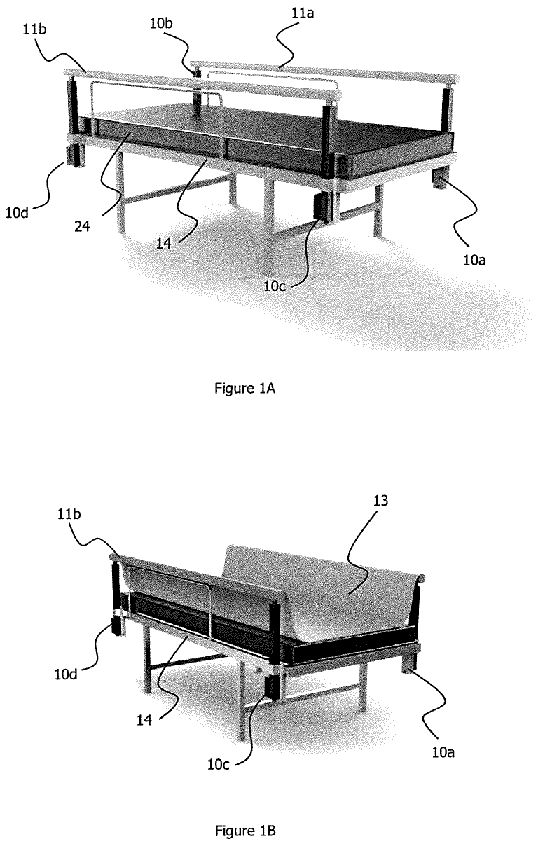

FIG. 1A shows a hospital bed 14. The hospital bed 20 includes a mattress 24 having a bed frame. The mattress is adjustable from a substantially flat configuration to a head elevated configuration.

The complete patient turning system comprises of a System Controller, a Lifting Apparatus and an Intelligent Sensor System.

### Patient Turning Apparatus

FIG. 1B shows a Patient Turning Apparatus. The Patient Turning Apparatus includes a sheet 13, a plurality of linear actuators 10a, 10b, 10c, 10d and a pair of connecting beams 11a, 11b. The actuators are connected to the System Controller 12 via a wired connection.

The sheet 13 includes a length L and a width W. The sheet is preferably sized and shaped to accommodate a wide variety of patients having various heights, weights and widths. Preferably, the length of the sheet is sized, shaped and configured to accommodate an immobile person from the top of the head to the mid-calf region. The sides of the sheet 13 are fixed to the first and second connecting beams 11a, 11b. Preferably, the length of the sheet extends from the top of the head of the immobile person to the mid-calf region of the immobile person. The sides of the sheet have means to facilitate its securement to anchorages provided on the connecting beams 11a, 11b. Preferably, the sides of the sheet are fitted with hook-and-loop fasteners such as a Velcro® strap.

The sheet 13 is thin, flexible and smooth and possesses ample tensile strength so that when spread over the top surface of the mattress, it will be safe and comfortable for the immobile person to lie upon. The sheet can be made from different materials such as some of textile materials, woven or otherwise, others of synthetic materials, and still others of paper or the like. Preferably, the sheet is made of an antibacterial and waterproof material.

Linear actuators are positioned on the left and right sides of the bed frame. Preferably, two actuators 10a, 10b are positioned on one longitudinal side of the bed frame and two actuators 10c, 10d are positioned on the other longitudinal side of the bed frame. The actuators are vertically and horizontally aligned.

Although the preferred embodiment has been described using linear actuators, other actuators may be used.

The connecting beams 11a, 11b have means to facilitate its securement to anchorages provided on the sheet 13. Preferably, the connecting beams 11a, lib are fitted with hook-and-loop fasteners such as a Velcro® strap.

As shown in FIG. 1C, the ends of the connecting beam 11a are fixed to longitudinal actuators 10a, 10b. Similarly, the ends of the connecting beam lib are fixed to the other longitudinal actuators 10c, 10d. Each actuator has means to facilitate its securement to anchorages provided on the connecting beams 11a, 11b. The attachment point of the actuators 10a, 10b, 10c, 10d and beams 11a and 11b has rotatable hinges which allows the top beam to move without breaking the beam. Preferably, the actuators are secured to the connecting beams by way of bolts and nuts.

As shown in FIG. 1D, each actuator has means to facilitate its securement to the bed frame. For example, each of the actuators 10a, 10b, 10c, 10d are fixed to the bed frame by means of fasteners such as clamps, plates or screws so as to be removable and replaceable. Preferably, the actuators 10a, 10b, 10c, 10d are fixed to the bed frame by means of a mounting bracket.

When in use, multiple actuators are engaged at one time. When engaged, the actuator is extended. When the actuator is extended, the sheet is tilted to the right, left, front or back. Thus, it is capable of repositioning the immobile person's body to relieve pressure from the occipital region, shoulder blades, the sacral region, and the calcaneal region.

To relieve pressure from the occipital region, the head and trunk of the immobile person are raised. Accordingly, the left side second actuator 10b and right side second actuator 10d are engaged.

To relieve pressure from the shoulder blades, the head and trunk of the immobile person are raised. Accordingly, the left side second actuator 10b and right side second actuator 10d are engaged.

To relieve pressure from the sacral region, the immobile person is turned either on his right or left side. Accordingly, the left side actuators 10a, 10b are engaged raising the first connecting beam 11a. Alternatively, the right side actuators 10c, 10d are engaged raising the second connecting beam 11b as shown in FIG. 1E. If none of the actuators have been engaged for two hours, the patient or healthcare provider may manually select which actuators to activate.

To relieve pressure from the calcaneal region, the foot of the immobile person is raised. Accordingly, the left side first actuator 10a and right side first actuator 10c are engaged.

### Intelligent Sensor System

The Intelligent Sensor System assists in the management of pressure sores. It provides information on interaction of the interface pressure between the immobile person and the surface with which they are in contact, for example, a mattress.

The Intelligent Sensor System comprises of a plurality of wearable articles 20, 30, 40, 50. The wearable articles 20, 30, 40, 50 are preferably adapted for detecting and/or measuring any changes in pressure forces applied to the immobile person. The wearable articles provide relevant data to the System Controller 12.

Each wearable article comprises of a sensor component comprising of at least two sensors and a main controller comprising of batteries, power circuits, a Wi-Fi™ and Bluetooth® combination module and a microcontroller. The main controller is placed in a casing.

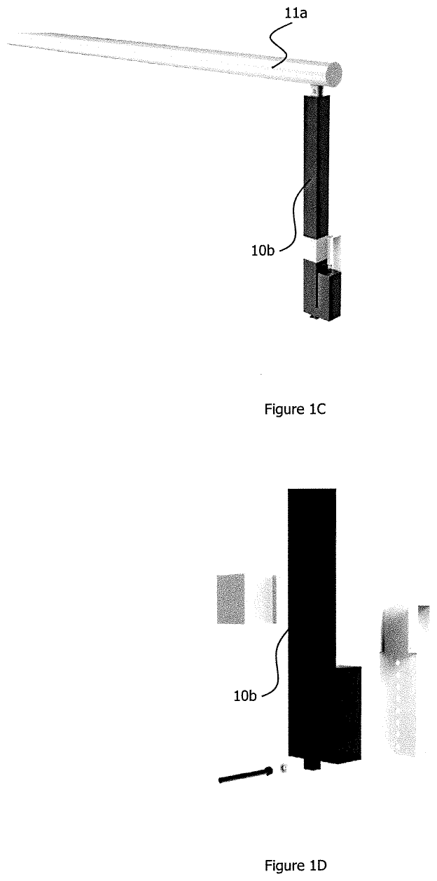

FIGS. 2A and 2B shows a wearable article in the form of a belt 20. The belt 20 has an inside surface 21 and an outside surface 22. The inside surface of the belt comprises of two Force Sensitive Resistor (FSR) sensors 7 which are horizontally aligned. Preferably, each FSR sensor 7 is located approximately 5 centimetres from the spinal line of an immobile person. The FSR sensors 7 are covered and protected from moisture by an antibacterial, waterproof fabric. The main controller is placed on the outer surface 22 of the belt 20. Preferably, the belt 20 is made of a stretchable material that is anti-bacterial and waterproof. The belt 20 is designed for a robust and comfortable fit around the waist of the immobile person. The belt 20 has a linking means that allows for easy and flexible adjustment of the belt according to the immobile person's waist size. An example of a linking means is a hook-and-loop fastener such as a Velcro® strap. Preferably, the linking means is a Velcro® strap. One Velcro® strap is positioned on one end of the inside surface 21 of the belt and another Velcro® strap is positioned on the opposing end of the belt on the outer surface 22 of the belt. When in use, the portion of the inside surface 21 of the belt having the FSR sensors 7 is in close contact to the sacrum region of the immobile person.

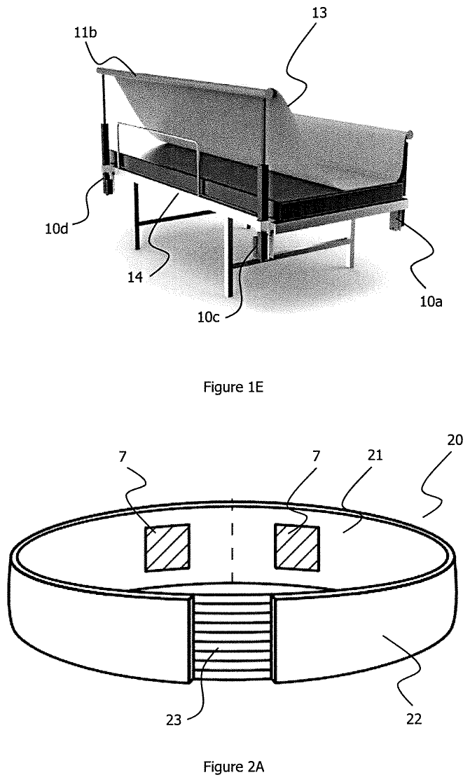

FIG. 3 shows a wearable article in the form of a headband 30. The headband 30 has an inside surface and an outside surface. The inside surface of the headband comprises of two FSR sensors 7 with are horizontally aligned. The FSR sensors 7 are covered and protected from moisture by an antibacterial, waterproof fabric. The main controller is placed on the outer surface of the belt. Preferably, the headband 30 is made of a stretchable material that is anti-bacterial and waterproof. The headband is designed for a robust and comfortable fit around the head of the immobile person. When in use, the portion of the inside surface of the headband having the FSR sensors 7 is in close contact with the back of the head of the immobile person.

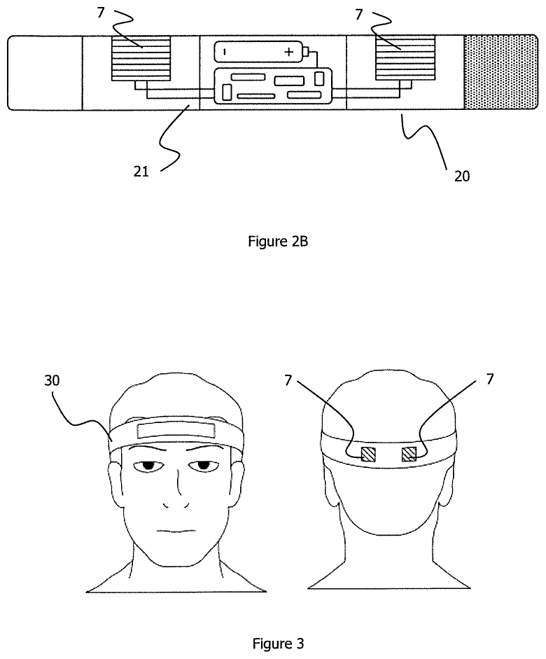

FIG. 4 shows a wearable article in the form of a vest 40. The vest 40 has a front 41 and back 42 portions. The back portion 42 of the vest comprises of two FSR sensors 7 which are horizontally aligned. The FSR sensors 7 are positioned on the vest 40 so that when in use, the FSR sensors 7 are placed in close contact with the shoulder blades of the immobile person. The FSR sensors 7 are covered and protected from moisture by an antibacterial, waterproof fabric. The main controller is placed on the front portion of the vest. Preferably, the vest 40 is made of a material that is anti-bacterial and waterproof.

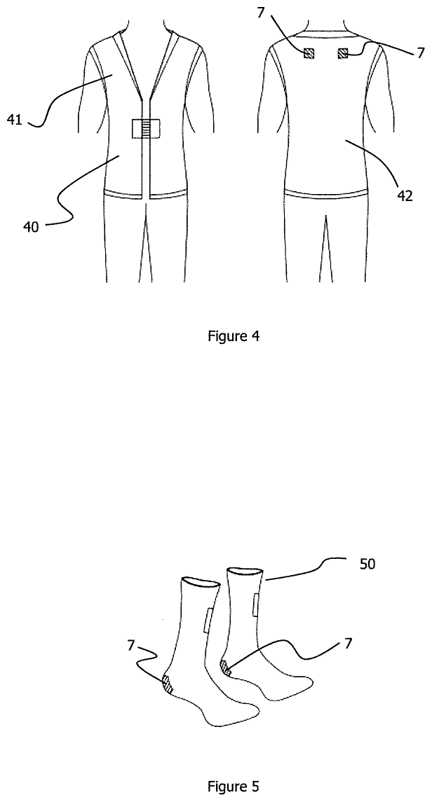

FIG. 5 shows a wearable article in the form of a sock. The heel portion of the sock comprises of a FSR sensor 7. The FSR sensor 7 is positioned on the sock 50 so that when in use, the FSR sensor 7 is placed in close contact with the calcaneus region of the immobile person. The FSR sensor 7 is covered and protected from moisture by an antibacterial, waterproof fabric. The main controller is placed on the leg portion of the sock 50. Preferably, the vest 50 is made of a material that is anti-bacterial and waterproof.

Each of the wearable articles is in communication with a System Controller 12 via a communication line. Examples of communication lines, but not limited to, are Bluetooth and Wi-Fi. Each of the wearable articles is preferably adapted to operate independently of other wearable articles. Each of the wearable objects can also function cohesively as a singular unit.

When each of the wearable articles is adapted to operate independently of each other, each of the cap, the vest, the belt, the socks functions independently and does not rely on the pressure values from the other wearable articles.

When the wearable articles function cohesively as a singular unit, the actuators will turn the immobile person until the unilateral pressure of values of one side of the body have reached below the accepted level of pressure. For example, if the immobile person is being turned on his left side, actuators 10c and 10d are engaged and will only disengage once all the pressure on the right side has been relieved.

The System Controller 12 is in communication with each of the wearable objects for receiving the response signals from the wearable articles and generating output signals responsive to the response signals to the actuators.

The pressure sensors are also able to provide feedback on the degree of tilt during repositioning. The actuators will stop when the pressure exerted on the sensors reaches about one half to one third of the full pressure of the body. This prevents the immobile person being overturned. If the actuators only stop when no pressure of the body is exerted on the sensors, this would mean that the immobile person would be lying flat on his/her face (overturning of the immobile person) as the actuators have overly extended.

As an alternative to wearable articles, the pressure sensors can be used alone meaning that it does not need to be attached to piece of clothing such as a belt, headband, vest or socks mentioned above. The sensor patch is connected to the System Controller 12. The sensor patch is to be used in circumstances where disposable items are preferred by the care facility. These sensor patches are especially advantageous for short-term immobile persons in care facilities as the sensor patches can be disposed of without having to wash for use on another immobile person.

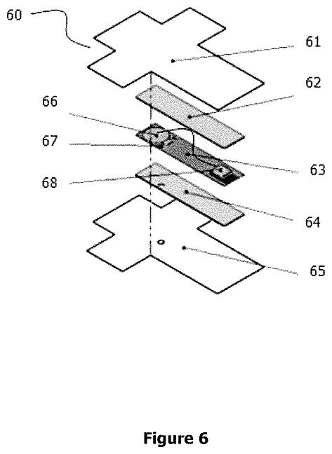

FIG. 6 shows a sensor patch 60. The sensor patch 60 has a backing layer 61 and an adhesive layer 65. In between the backing layer 61 and an adhesive layer 65 are polyethylene foam 63, pressure sensor 66, moisture/temperature sensor 67, microcontroller 68, flex PCB 63 and another polyethylene foam 65.

In an embodiment, multiple sensor patches 60 are embedded in the turning sheet 13. The immobile person will then no longer require the use of wearable articles for the prevention and relief of pressure sores.

Each sensor patch 60 is in communication with the System Controller 12 via a communication line. Examples of communication lines, but not limited to, are wires, Bluetooth and Wi-Fi. The multiple sensor patches 60 functions cohesively as a singular unit. The actuators will turn the immobile person until the unilateral pressure of values of a certain region of the body has reached below the accepted level of pressure. For example, if the immobile person is being turned on his left side, actuators 10c and 10d are engaged and will only disengage once all the pressure on the right side has been relieved.

The wearable articles and sensor patches are adapted to detect any changes in pressure applied to the immobile person so as to generate a feedback signal when pressure forces exceed a certain predetermined threshold value. As used herein, the term “force” includes any force to which the top surface may be subjected to including pressure forces, compressive forces, tensile forces, resonance, vibrations, thermal action, or other process forces. The above-identified forces are applied in a direction where the wearable object substantially parallel to the immobile person.

Although the embodiments have been described using Force Sensitive Resistors, other sensors that can detect physical pressure, squeezing and weight can be used.

### System Controller

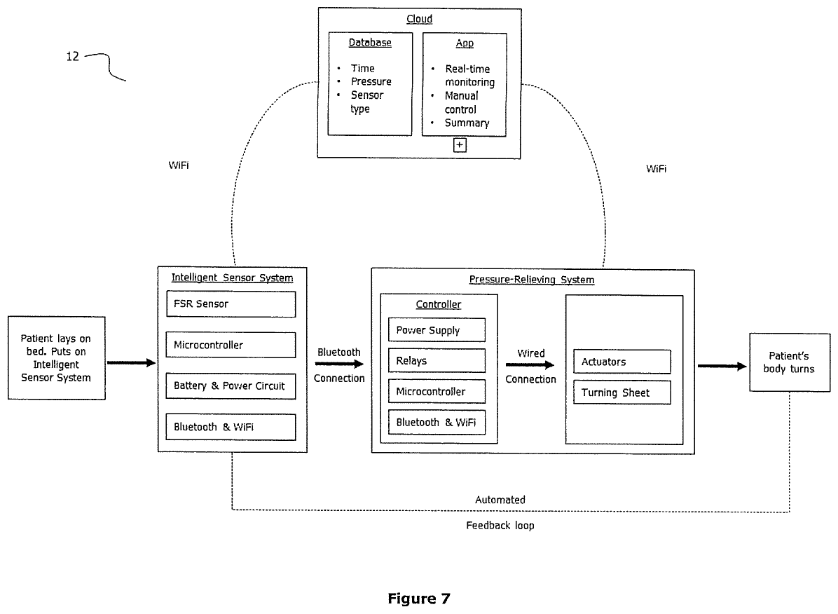

FIG. 7 shows a System Controller 12. The System Controller 12 preferably includes a microcontroller, communication module e.g. Bluetooth and/or Wi-Fi module, and a relay for selectively activating one or more of the actuators in the Pressure Relieving System. The system controller 12 may be located anywhere so long as it is in communication with the Intelligent Sensor System. In a preferred embodiment, the System Controller 12 is located on the bed frame.

The System Controller 12 is adapted to receive response signals from the Intelligent Sensor System. The System Controller 12 processes the response signals to determine whether the signals indicate whether the pressure has exceeded the threshold limit on any of the wearable article. If the threshold limit of the pressure force has exceeded is detected at one or more wearable article, the System Controller 12 transmits output signals for engaging the respective actuators.

Table 1 below shows the correlation between the response signal sent by the wearable article and the actuators that will be engaged:

TABLE 1

| | | Actuator | | | |
|---|---|---|---|---|---|
| | | 10a | 10b | 10c | 10d |
| Response | Headband | | x | | x |
| Signal from | Belt | | | | |
| Wearable | Left | x | x | | |
| Article | Right | | | x | x |
| | Vest | | x | | x |
| | Socks | x | | x | |

Table 2 below shows the correlation between the response signal sent by the region of the sensor patch in the turning sheet and the actuators that will be engaged:

TABLE 2

| | | Actuator | | | |
|---|---|---|---|---|---|
| | | 10a | 10b | 10c | 10d |
| Response | Occipital Region | | x | | x |
| Signal from | Sacral Region | | | | |
| Sensor Patch | Left | x | x | | |
| | Right | | | x | x |
| | Shoulder Blades | | x | | x |
| | Calcaneal Region | x | | x | |

When the actuators are engaged, the System Controller 12 stops the actuators from moving once the pressure values have reached an acceptable predetermined threshold value. Preferably, the actuators will stop moving when the pressure reaches about half to one third of the full pressure value.

The System Controller 12 uses one or more software applications stored therein capable of receiving feedback signals from the wearable articles, comparing the feedback signals with data stored in its memory, and generating a series of output signals for transmission to the actuators. Upon receiving the output signals, the one or more actuators are actuated for repositioning of the immobile person.

The System Controller 12 may notify medical personnel when sensed pressure forces are greater than a predetermined threshold value that requires interaction of medical personnel and/or personal inspection of the immobile person.

The System Controller 12 is able to tailor turn intervals, duration of turn, and direction of turn based on the input of the immobile person and/or medical personnel.

In another example, the system can be tailored to turn one side of the immobile person only. This is important when the immobile person has a sacral sore and a right hip sore and requires turning to the left side position only.

Additionally, it is possible to turn off the Intelligent Sensor System. Once turned off, the actuators will only be engaged every two hours following the same sequence prior to being turned off. This minimizes sleep disturbances during the night.

The Intelligent Sensor System and System controller is connected to a mobile application which displays the pressure values real time. The mobile application is able to produce a summary of pressure on a particular body surface area of the immobile person. This mobile application also allows manual control of the actuators.

### EXAMPLE

The following Example is when the automated turning system is in use. This Example does not limit the invention, the scope of which is set out in the appended claim.

Immobile person A has a Grade 3 sacral pressure ulcer, a Grade 2 heel pressure ulcer and a Grade 3 occipital pressure ulcer.

The sequence and frequency of turns are dependent on various inputs such as:

the severity of the pressure ulcer;
the location of the ulcer where sacral ulcers will always be top priority due to its location and potential for rapid deterioration; and
the wearable article(s) worn by the immobile person.

An example of the turning schedule based on immobile person A is as follows:

Time 0 min: Both left side actuators 10a and 10b are engaged.
Time 15 min: Both left side actuators 10a and 10b are disengaged.
Time 20 mins: Both top actuators 10b and 10d are engaged.
Time 25 mins: Both top actuators 10b and 10d are disengaged.
Time 30 mins: Both right side actuators 10c and 10d are engaged.
Time 45 mins: Both right side actuators 10c and 10d are disengaged.
Time 50 mins: Both top actuators 10b and 10d are engaged.
Time 55 mins: Both top actuators 10b and 10d are disengaged and both bottom actuators 10a and 10c are engaged.
Time 60 mins: Both bottom actuators 10a and 10c are disengaged and both left side actuators 10a and 10b are engaged.

When the Intelligent Sensor System is turned off during the night, the actuators will be engaged every two hours following the same sequence as above.

1. An automated turning system for an immobile person comprising:
a plurality of pressure sensors for measuring pressure forces applied to the immobile person and generating response signals that correspond to said pressure forces applied to said immobile person;
a system controller adapted to receive said response signals from said pressure sensors and generate a plurality of output signals in response to each of said response signals;
a plurality of actuators being adapted to receive said output signals from said system controller; and
a lifting attachment comprising a sheet and a pair of connecting beams, each beam being connected to ones of said plurality of actuators for raising a portion of said sheet to a tilted position, wherein, in use, the immobile person is positioned atop said sheet;
wherein said system controller receives said response signals for detecting whether a pressure threshold value for said immobile person has been exceeded for any of said sensors, and in turn, said system controller transmits said output signals to said actuators for engagement.

2. The system as claimed in claim 1, further comprising a bed with bed frames, wherein the plurality of actuators is mounted on the bed frame.

3. The system as claimed in claim 1, wherein the plurality of actuators is engaged if a predetermined threshold value of the pressure sensor has not been exceeded for any of said plurality of sensors for a preset interval.

4. The system as claimed in claim 3, wherein said preset interval is two hours.

5. The system as claimed in claim 1, wherein each of said pressure sensor operates independently of one another.

6. The system as claimed in claim 1, wherein said plurality of pressure sensors operates cohesively as a singular unit.

7. The system as claimed in claim 1 wherein, in use, said pressure forces are detected in any one or more of the occipital region, the thoracic spine region, the shoulder blades region, the sacral region and the calcaneal region.

8. The system as claimed in claim 1 wherein the pressure sensors are embedded into said sheet.

9. The system as claimed in claim 1 wherein the pressure sensors are embedded into a disposable patch.

10. The system as claimed in claim 1 wherein the pressure sensors are embedded into a wearable article.

11. The system as claimed in claim 10 wherein said wearable article is any one or more of a headband, vest, belt or socks.

12. The system as claimed in claim 1 wherein said plurality of pressure sensors are wirelessly connected to the system controller.

13. The system as claimed in claim 1 wherein said plurality of actuators are connected to the system controller via a wired connection.

14. The system as claimed in claim 1 wherein said plurality of pressure sensors are Force Sensitive Resistors.

15. A bed comprising the automated turning system as claimed in claim 1.
